(19) United States
(12) Patent Application Publication
(10) Pub. No.: US 2011/0137417 A1
(43) Pub. Date: Jun. 9, 2011

(54) Multiple wafer cortical bone and cancellous bone allograft with cortical pins

(75) Inventor: John W. Lee, San Antonio, TX (US)

(73) Assignee: Transplant Technologies of Texas, San Antonio, TX (US)

(21) Appl. No.: 12/631,489

(22) Filed: Dec. 4, 2009

(51) Int. Cl. A61F 2/28

(52) U.S. Cl. 623/16.11

(57) ABSTRACT

A bone allograft and cortical pin for inserting into a surgically altered site of a human. The cortical pin is cylindrically shaped and has a thick middle diameter surrounded by two smaller end portions. The allograft has two end cortical wafers with small canals to receive the small ends of the cortical pin. At least one cancellous wafer is disposed adjacently between the end cortical bone wafers. A plurality of cancellous and/or cortical wafers may be inserted between the end cortical wafers to form the allograft. The wafers inserted between the end cortical wafers have larger canals to receive the thick diameter of the cortical pin. The size of the allograft may be adjusted as desired by either adding or removing inner wafers, or adjusting the size of the inner wafers of the allograft. The allograft and cortical pin are inserted parallel to the plane of insertion into the human.

### BACKGROUND OF THE INVENTION

1. Field of the Invention

The present invention relates to a bone allograft for implantation into a surgically altered area or site of a human, and more specifically, a bone allograft having a plurality of cortical bone and cancellous bone segments or wafers articulating with one another through a series of tabs and notches therein. A cortical pin is inserted through the widths of the respective bone wafers to form the allograft. The cortical pin is substantially cylindrically shaped, having thin diameter along its end portions and a thick middle diameter, thereby creating a shoulder to absorb stress placed on the allograft by insertion into the surgically altered site and to channel the stress throughout the cortical bone rather than the cancellous bone portion of the graft.

2. Description of the Related Art

A common problem many people encounter either as they get older or through injury is the collapsing of inter-vertebral discs. As the adjacent vertebrae collapse together, nerves are pinched causing further pain to the person as the vertebrae collapse upon one another. It is common to stabilize collapsing vertebrae by placing heterogeneous bone allografts from a human donor intervertebrally. Ideally, hard cortical bone would be retrieved from a donor and transplanted into a surgically altered site. Specifically, it would be optimal to insert hard cortical bone allografts between vertebrae to allow the cortical bone to fuse with the superior and inferior vertebrae to stabilize the vertebrae and provide relief to the patient.

However, because of limitations naturally placed on the tissue retrieving process by the human anatomy, and the eligible donor pool, the cortical bone segments of a donor which are desirable to retrieve for transplantation are fairly limited. The suitable cortical segments can only be retrieved from the shafts of the long bones of the human body, making it difficult if not impossible to retrieve a sufficient volume of single cortical bone segments or multiple segments from a donor of sufficient size and shape to insert inter-vertebrally or otherwise between bones or bone segments of a patient.

Moreover, the healing process wherein the heterogeneous cortical bone is incorporated into the native bone tissue makes it impractical to insert an allograft made completely of cortical bone. This is because fusion of cortical tissue to native bone of a patient occurs slowly over a long period of time by a reverse mechanism wherein osteoclasts break down portions of the implanted cortical tissue, creating canals through the cortical tissue to allow the body to vascularize the bone through the channels or canals and allow the native blood to supply rebuilding bone molecules to the cortical bone.

The cortical bone is initially weakened in this process, and is later strengthened as the heterogeneous cortical bone segment or segments are fused to the native bone of the patient. This process can take years to complete. Therefore, the stability ultimately provided by cortical bone segments is not provided in the interim between post-operation and substantial completion of the reverse mechanism healing process. Thus, a cortical bone implant cannot bear loads placed on it by the body by itself until it is stabilized within the surgically altered site by fusing to the native bone.

Cancellous bone fuses to native bone tissue much more quickly. Cancellous bone is very spongy, containing many vascularized canals. The patient's body sends native blood supply to the cancellous bone very quickly and allows the cancellous bone to vascularize and incorporate quickly into the native bone. However, cancellous bone is very weak, and cannot bear significant loads placed on it by the body by itself.

It is therefore desirable to construct a bone allograft having segments or wafers of cortical bone and segments or wafers of cancellous bone so that the allograft can initially fuse to native bone tissue via fusion with the cancellous wafers, thus providing initial stability to the allograft to allow the prolonged fusion of the cortical bone wafers to the native bone tissue. It is further desirable to construct the allograft in such a way that it absorbs forces placed on it during insertion into the surgically altered site without breaking apart. It is further desirable to construct the allograft in such a way as to allow interspersal of cortical bone and cancellous bone wafers adjacent one another so that larger bone allografts can be utilized in transplantation. It is further desirable to construct a cortical bone pin that can absorb insertion force during implantation of the allograft. It is further desirable to construct the allograft in such a way that it absorbs forces placed on it during the incorporation of the allograft into the recipient's anatomy.

There exists in the prior art bone allografts for insertion and/or fusion into the spinal column wherein the bone allograft has cortical bone wafers and cancellous bone wafers. However, bone allografts in the art suffer from several drawbacks. Some allografts do not utilize pins to hold the bone wafers together. Such allografts can easily break apart during insertion. Other allografts have pins inserted through the wafers of cortical and cancellous bone. However, in many instances the pins are not inserted completely through the allograft. Moreover, the pins are typically thin, cylindrical, straight, and of a uniform small diameter, lending to a tendency to be easily dislodged from the allograft if the allograft is jarred or encounters a blunt force such as the forces necessary to insert the allograft into the patient.

Another problem associated with allografts in the art using the slender cylindrical pins is that such allografts are inserted into the surgically altered area such that the length of the pin bears the insertion load asserted on the allograft by a surgical mallet or other surgical device typically used to insert a bone allograft inter-vertebrally or otherwise between bone wafers. In other words, the bone pin is exposed across, or perpendicular to the insertion plane as opposed to with, or parallel to the plane of insertion.

By inserting the allograft such that the pin is perpendicular to the plane of insertion, each individual bone wafer of the allograft is likewise perpendicular to the plane of insertion. By exposing the allograft in such a way, the insertion force is asserted not only across the length of the bone pin, but also directly upon each bone wafer. Therefore, each strike of the mallet or other surgical tool must be sustained substantially equally by each cortical and cancellous bone wafer in order to keep the bone pin from breaking and the allograft from coming apart. This is nearly impossible to accomplish, and it is therefore common for such allografts to fall apart during or shortly after insertion. This problem is exasperated by the fact that the bone pins—if used at all by the prior art—are thin, straight cylindrical rods, resembling a straight pin. The construction of these pins does not allow the pin to successfully absorb the force of insertion asserted on the pin by a mallet or other surgical instrument. Thus, the pins break and the allograft comes apart.

### BRIEF SUMMARY OF THE INVENTION

The present invention is different than the prior art. The bone allograft of the present invention is held together by a bone pin made of cortical bone. The cortical pin is made of a single piece of cortical bone and is substantially cylindrical, and shaped similar to a rolling pin. The cortical pin has a thick middle diameter which corresponds to and is inserted within canals in the inner bone wafers of the allograft. The cortical pin has diameters smaller than the middle diameter along its end portions.

The smaller diameter end portions of the cortical pin correspond to and are inserted within the end cortical bone wafers of the allograft. The junctions of the small end portions of the cortical pin with the thick middle diameter of the cortical pin create a shoulder on each side of the thick middle diameter. The shoulders aid in absorbing the brunt of the force asserted on the allograft by a surgical mallet or other appropriate medical device during insertion of the allograft.

The allograft is comprised of two end cortical bone segments or wafers. The cortical end wafers have at least one hole or tubular canal extending through the width of the wafers. The canal is of a diameter sufficient to receive the end portions of the cortical pin snugly, but too small in diameter to receive the thick middle diameter of the cortical pin. At least one cancellous bone wafer is disposed adjacently between the cortical end wafers. Where a small bone allograft is desired, as few as one cancellous bone wafer may be disposed adjacently between the cortical end wafers. The size of the allograft desired can be accommodated by either adding cancellous bone wafers adjacent one another between the cortical end wafers, or cutting wider cancellous wafers and inserting them between the end cortical wafers.

However, if too many cancellous bone wafers are placed adjacent one another such that the width of the adjacent cancellous segments become too wide, or if a single cancellous bone member is created too wide, the cancellous wafer or wafers will simply collapse or crush between the cortical end wafers during insertion of the allograft and/or during remodeling or incorporation of the allograft. It is therefore desirable—especially where larger allografts are required—to have inner cortical bone wafers interspersed between adjacent cancellous bone wafers to add structural integrity and additional load-bearing support to the inner portion of the allograft to prevent such crushing.

In fact, it is desirable to have each cancellous bone member created to be approximately eight millimeters wide or less to reduce the risk of crushing or collapsing during insertion or the pending remodeling. Alternatively, if multiple thinner cancellous wafers are placed adjacent one another in the allograft, it is desirable to have a cortical wafer interposed between such multiple thinner cancellous wafers such that the total width of the cancellous wafers adjacent one another is approximately eight millimeters or less. Each wafer disposed between the end cortical wafers has one or more tubular canal(s) disposed through the wafer across the width thereof. The canal is of sufficient size to snugly receive the middle diameter of the cortical pin.

In one aspect of the invention, all of the wafers are disposed side by side such that sides of the wafers which are adjacent one another are substantially flat. However, in another aspect of the present invention, the wafers comprise a trough and shelf, or tab and groove configuration to interlock adjacent wafers. This incorporated feature serves two purposes. First it aids is absorbing insertion forces associated with surgically placing the allograft. Second, it provides additional strength to the composite allograft to decrease the likelihood of the allograft fracturing during the remodeling process. One of the end cortical wafers has a tab along the side of the cortical wafer that is disposed on the internal side of the allograft. A groove is disposed along the adjacent side of an adjacent cancellous wafer. The groove is formed to snugly receive the tab of the end cortical wafer. On the side of the cancellous wafer opposite the groove is a tab substantially the same as the tab on the end cortical wafer.

The wafer adjacent the cancellous wafer—whether cancellous or cortical—has a groove to receive the tab of the cancellous wafer, and a tab on the side opposite the groove which will be inserted into a groove of an adjacent wafer. Each internal wafer has the tab and groove configuration such that each groove receives the tab of the adjacent wafer. The other end cortical wafer has a groove for receiving the tab of an adjacent cancellous wafer.

The allograft of the present invention is inserted into the surgically altered area of the patient with its assembled sections laying perpendicular to the way the other allografts in the art are inserted. Specifically, the allograft is inserted such that the cortical pin runs with, or parallel to the plane of insertion as opposed to perpendicular to the plane of insertion. By inserting the allograft parallel to the plane of insertion, one end cortical wafer is receiving the direct impact from the surgical mallet or other insertion device, and the other end cortical wafer is receiving the transferred impact from the cortical pin. Moreover, insertion of the allograft parallel to the plane of insertion aids in preventing fracturing and reducing stress placed on the allograft during the remodeling process.

The tab and groove configuration of the wafers allows the wafers to interlock with one another to add stability of the allograft during insertion and especially during remodeling. Furthermore, the tab of the end cortical wafer provides an elevated shelf which is adjacent the shoulder formed by the junction of the end portion and the middle diameter of the pin. As the wafer is impacted by the mallet during insertion, the energy is transferred through the allograft by the cortical pin and is absorbed by the end cortical wafer and the end portion of the cortical pin disposed therein.

Specifically, the configuration of the cortical pin allows the energy from the mallet to be transferred from the thin end portion disposed within the end cortical wafer that receives the direct impact from the surgical mallet to the thick middle diameter of the cortical pin. The energy is then displaced through middle diameter of the bone pin to the tab of the opposite end cortical wafer, where the shoulder formed by the junction of the end portion and middle diameter of the cortical pin abuts the opposite end cortical wafer. Thus, the construction of the cortical pin and the allograft, in addition to the alignment of the allograft in relation to the plane of insertion allow for optimal energy transfer and displacement through the allograft to minimize the risks of the cortical pin breaking and/or the allograft otherwise coming apart.

### BRIEF DESCRIPTION OF THE DRAWINGS

FIG. 1 is a perspective exploded view of a three-wafer allograft of the present invention;

FIG. 2 is a perspective exploded view of a five-wafer allograft of the present invention;

FIG. 3 is a perspective partially exploded view of a three-wafer allograft of the present invention;

FIG. 4 is a perspective view of a three-wafer allograft of the present invention;

FIG. 5 is a perspective view of a five-wafer allograft of the present invention;

FIG. 6 is a perspective exploded view of a three-wafer allograft of the present invention;

FIG. 6A is a sectional view of a three-wafer allograft of the present invention along line 6-6 of FIG. 6;

FIG. 7 is a perspective exploded view of a four-wafer allograft of the present invention;

FIG. 7A is a sectional view of a four-wafer allograft of the present invention along line 7-7 of FIG. 7;

FIG. 8 is a perspective exploded view of a five-wafer allograft of the present invention;

FIG. 8A is a sectional view of a five-wafer allograft of the present invention along line 8-8 of FIG. 8;

FIG. 9 is a front perspective view of multi-wafer allografts of the present invention having two cortical pins;

FIG. 10 is a front perspective view of multi-wafer allografts of the present invention having two cortical pins;

FIG. 11 is a top view of an anterior lumbar inner fusion allograft of the present invention;

FIG. 12 is a side sectional view of an allograft of the present invention having two cortical pins; and

FIG. 13 is a side exploded view of the tab and groove configuration of the internal wafers of the allograft of the present invention.

### DETAILED DESCRIPTION OF THE INVENTION

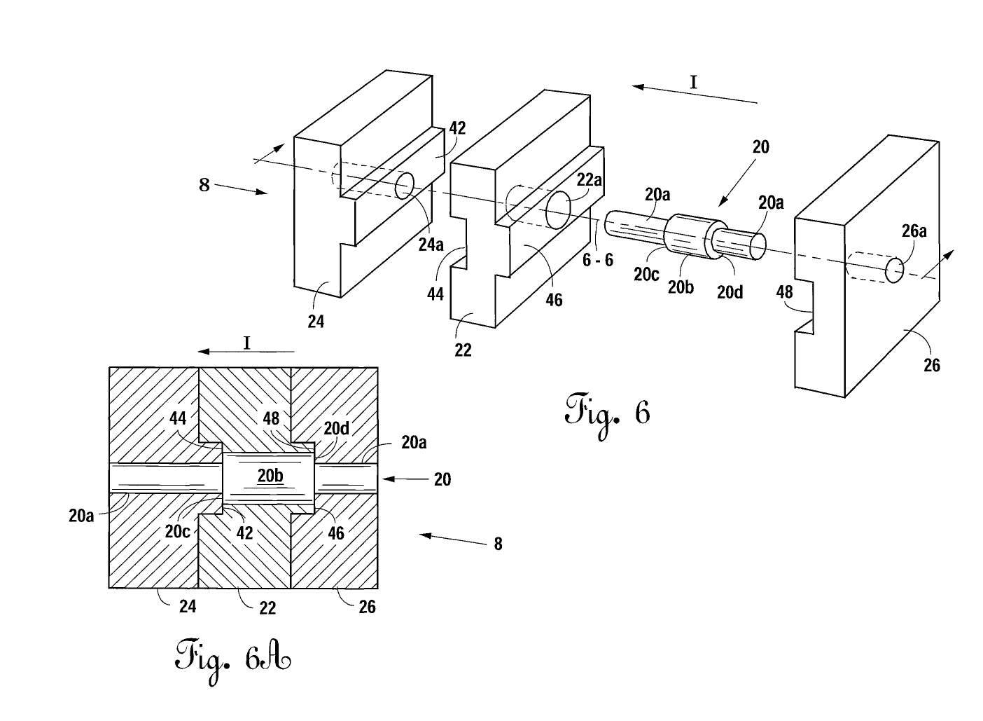

Referring to FIGS. 1-5, bone allografts 8 of one embodiment of the present invention are disclosed. In FIGS. 1-8 herein reference arrow “I” refers to the plane and direction of insertion of the allograft 8 of the present invention into the surgically altered site of the human (not shown). Referring to FIGS. 1, 3 and 4, an embodiment depicting a three-wafer allograft 8 is disclosed. The three-wafer allograft 8 has two end wafers 14. Wafers 14 are cortical bone wafers. A cancellous bone wafer 12 is disposed adjacently between cortical wafers 14.

Each cortical wafer 14 and cancellous wafer 12 has at least one canal 14a and 12a, respectively. The canals 14a and 12a are all substantially aligned with one another such that cortical pin 10 is inserted through the canals 14a and 12a of the cortical wafers 14 and cancellous wafers 12, respectively, to form the allograft 8. Cortical pin 10 is preferably made of cortical bone, and is constructed of a single piece of cortical bone. However, it should be understood that alternatively all cortical pins 10, 20, 30, 38 and 40 can be made of multiple pieces and may constitute any combination of cortical and cancellous bone. Moreover, while shown in FIGS. 1-5 as having two separate cortical pins 10, and a corresponding set of canals 14a and 12a, it should be understood that the allograft of the present invention could comprise one cortical pin 10 or multiple cortical pins 10 as shown. Moreover, it should be understood with respect to the embodiments disclosed in FIGS. 6 through 8A that multiple cortical pins 20, 30 or 40 could be inserted into the allograft 8.

As shown in FIGS. 1, 3 and 4, cortical pin 10 is a straight cylindrical rod of a substantially uniform diameter. However, it is also desirable for cortical pin 10 to have a construction similar to a typical rolling pin, such as the construction shown as cortical pin 20 in FIGS. 6 and 6A. In such an embodiment, canal 12a would be of a sufficient size to snugly receive the enlarged middle diameter 20b of the cortical pin 20. Canals 14a would be of sufficient size to snugly receive the smaller end portions 20a of the cortical pin 20, but too small to receive the middle diameter 20b.

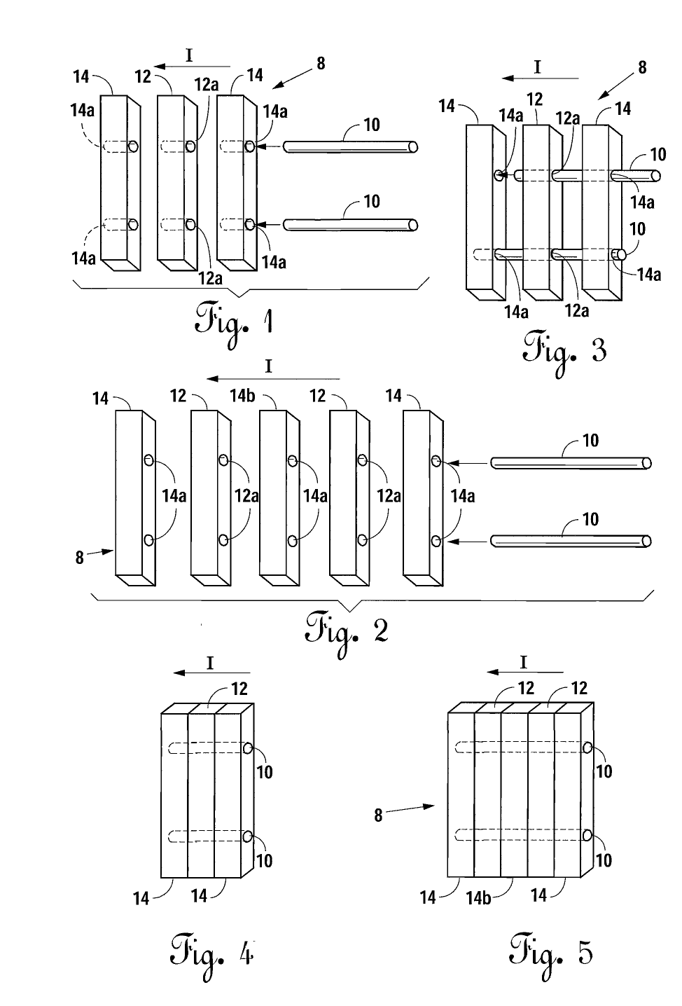

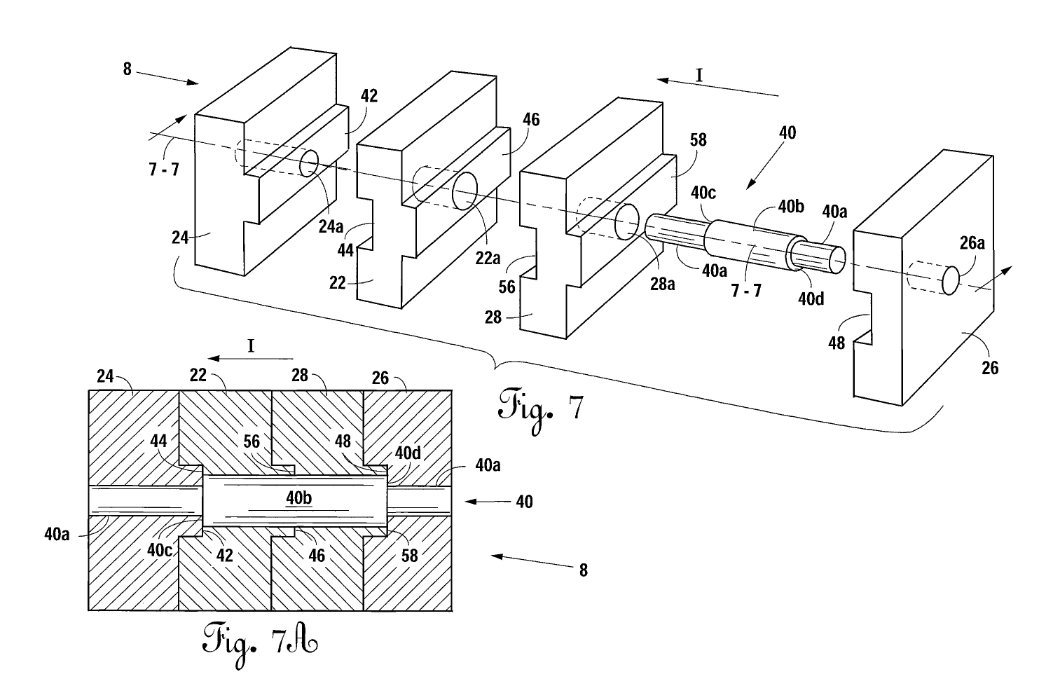

Referring to FIGS. 2 and 5, a five-wafer allograft 8 of another embodiment of the present invention is disclosed. Like FIGS. 1, 3 and 4, wafers 14 are cortical wafers and wafers 12 are cancellous wafers. The allograft 8 has cortical wafers 14 on each end of the allograft. Cancellous wafers 12 are disposed adjacent the cortical wafers 14 on the ends of the allograft 8. An internal cortical wafer 14b is disposed adjacently between the two cancellous wafers 12. The internal cortical wafer 14b adds structural strength to the allograft 8. By having a hard internal cortical wafer 14b between the cancellous wafers 12, energy is dispersed through the allograft 8 during insertion from one soft cancellous wafer to the hard internal cortical wafer 14b before the energy continues to the second cancellous wafer 12. The addition of internal cortical wafer 14b aids in preventing collapsing or crushing of the cancellous wafers 12 during insertion of the allograft 8.

Each cortical wafer 14, 14b and cancellous wafer 12 has at least one canal 14a and 12a, respectively. The canals 14a and 12a are all substantially aligned with one another such that cortical pin 10 is inserted through the canals 14a and 12a of the cortical wafers 14, 14b and cancellous wafers 12, respectively, to form the allograft 8. Cortical pin 10 is preferably made of cortical bone, and is constructed of a single piece of cortical bone. However, it should be understood that alternatively all cortical pins 10, 20, 30, 38 and 40 can be made of multiple pieces and may constitute any combination or cortical and cancellous bone.

As shown in FIGS. 2 and 5, cortical pin 10 is a straight cylindrical rod of a substantially uniform diameter. However, it is also desirable for cortical pin 10 to have a construction similar to a typical rolling pin, such as the construction shown as cortical pin 40 in FIGS. 8 and 8A. In such an embodiment, canals 12a of cancellous wafers 12 as well as canal 14a of internal cortical wafer 14b would be of a sufficient size to snugly receive the enlarged middle diameter 40b of the cortical pin 40. Canals 14a of cortical wafers 14 would be of sufficient size to snugly receive the smaller end portions 40a of the cortical pin 40, but too small to receive the middle diameter 40b.

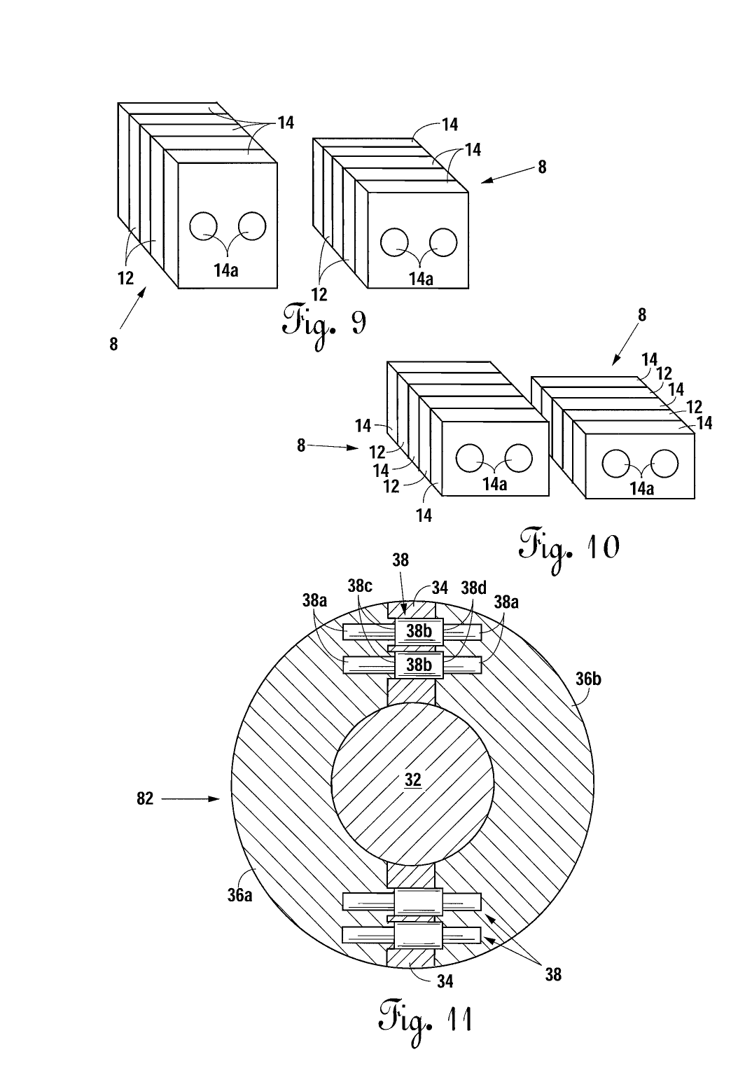

The wafers 14, 14b and 12 in FIGS. 1 through 5, in addition to the wafers shown in FIGS. 6 through 12 are shown as being substantially columnar. However, it should be understood that the wafers disclosed herein could be of any shape and any size desirable to size and shape the allograft 8 to meet the needs of the patient's surgically altered site. The wafers disclosed herein have a width and a length longer than the width, but otherwise are not intended to be restricted to a particular shape or size.

Referring now to FIGS. 6 through 8A, allografts 8 of another embodiment of the present invention having tab and groove configurations are disclosed. Referring to FIGS. 12 and 13, the tab and groove configuration of the internal cancellous wafer 12 and internal cortical wafer 14b are generally shown. Referring to FIGS. 6 and 6A a three-wafer allograft 8 is disclosed. The allograft 8 has an end cortical wafer 24. The end cortical wafer 24 has a tab 42. The tab 42 is disposed along the internal face of the cortical wafer 24, and extends the length thereof. The tab 42 provides a shelf for receiving the shoulder 20c of cortical pin 20. Cortical wafer 24 has a canal 24a that is of sufficient size to snugly receive one end portion 20a of the cortical pin 20, but is too small to receive the middle diameter 20b.

Adjacent cortical wafer 24 is a cancellous wafer 22. Along the side adjacent tab 42 is a corresponding groove 44. The groove 44 extends the length of the cancellous wafer 22. The groove 44 is sized to snugly receive the tab 42, thereby allowing cortical wafer 24 to interlock with cancellous wafer 22. On the side of cancellous wafer 22 directly opposite the groove 44 is a tab 46 which extends the length of cancellous wafer 22. Tab 46 is substantially the same size and shape as tab 42, although some variation of size and shape of the tabs of the wafers discussed herein is acceptable so long as the groove of the adjacent wafer is sized and shaped appropriately to snugly receive the tab 46. Cancellous wafer 22 has a canal 22a. The canal 22a is of sufficient size to snugly receive the middle diameter 20b of cortical pin 20.

Adjacent cancellous wafer 22 is an end cortical wafer 26. Cortical wafer 26 has a groove 48 corresponding to tab 46 of cancellous wafer 22. The groove 48 extends the length of the cortical wafer 26, and is sized to snugly receive the tab 46 of the cancellous wafer 22, thereby allowing cortical wafer 26 and cancellous wafer 22 to interlock. Cortical wafer 26 has a canal 26a which is substantially the same as canal 24a. Canal 26a snugly receives the other end portion 20a of cortical pin 20, but is too small to receive middle diameter 20b.

Referring to FIG. 6A, in constructing the allograft 8, cancellous wafer 22 is first placed on the cortical pin 20 by inserting an end 20a through the canal 22a, and sliding the cancellous wafer 22 onto the middle diameter 20b. Next, cortical member 24 slides onto the end portion 20a of the cortical pin 20 such that shoulder 20c rests on tab 42 once tab 42 is inserted into groove 44. Finally, cortical member 26 slides onto the opposite end portion 20a of the cortical pin 20 such that shoulder 20d rests within tab 46, which is in turn surrounded by cortical wafer 26 due to the groove 48 receiving tab 46 of cancellous wafer 22.

As the allograft 8 is inserted into the surgically altered site of the patient (not shown) in the plane of insertion I, a surgical mallet (not shown) or other appropriate medical/surgical device (not shown) is used to strike the allograft 8 in the direction of the plane of insertion I. Cortical wafer 26 and the end portion 20a of cortical pin 20 disposed within cortical wafer 26 absorb the initial energy imparted on the allograft 8. The energy imparted on the end portion 20a of the cortical pin 20 is transferred through shoulder 20d, which is surrounded by the stronger cortical bone tissue of cortical wafer 26, and into middle diameter 20b. From middle diameter 20b, the energy is transferred through the shoulder 20c of the cortical pin 20, which is abutted against the hard cortical tab 42 of cortical member 24, thereby transferring the energy from cortical pin 20 to cortical wafer 24 and the end portion 20a of cortical pin 20 disposed within cortical wafer 24.

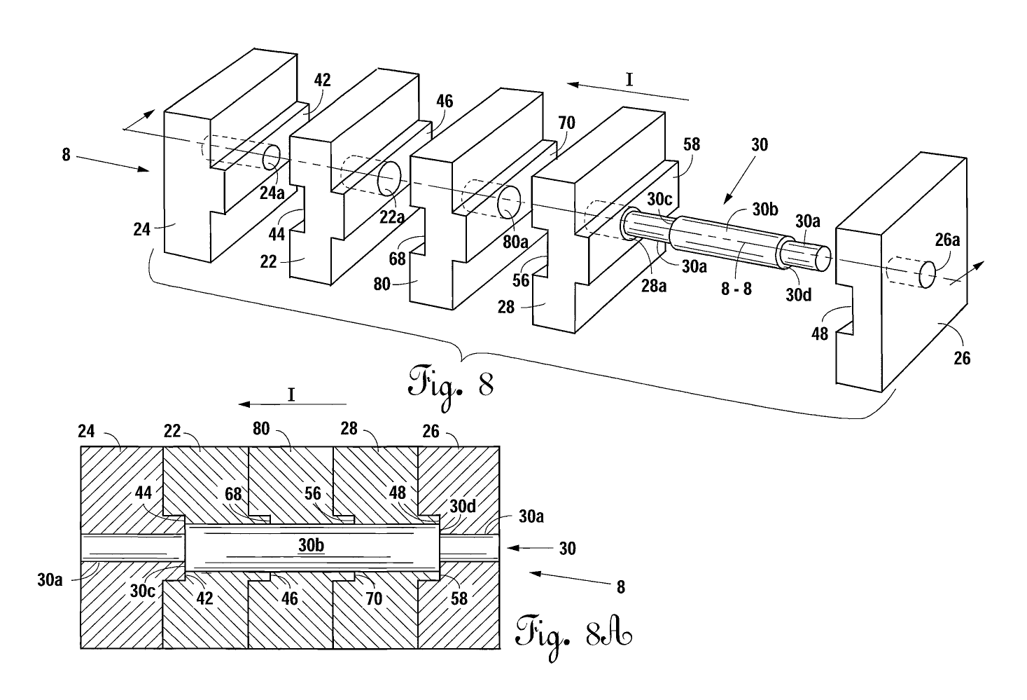

Referring to FIGS. 7 and 7A, an embodiment of the allograft 8 of the present invention comprising four wafers is disclosed. The allograft 8 has end cortical wafers 24 and 26 as described with respect to FIGS. 6 and 6A hereinabove. The allograft 8 also has cancellous wafer 22 as described with respect to FIGS. 6 and 6A hereinabove. However, as shown in FIG. 7, a second cancellous wafer 28 is disposed adjacently between cancellous wafer 22 and cortical wafer 26. Cancellous wafer 28 has a groove 56 which extends the length of cancellous wafer 28 and snugly receives tab 46 of cancellous wafer 22. On the side of cancellous wafer 28 opposite groove 56 is a tab 58 extending the length of cancellous wafer 28, which is snugly received by groove 48 of cortical wafer 26. Cancellous wafer 28 has a canal 28a which is sufficiently sized to snugly receive middle diameter 40b of cortical pin 40. Cortical pin 40 is substantially the same as cortical pin 20 except that its middle diameter 40b is elongated sufficiently to snugly reside within cancellous wafers 22 and 28.

Referring to FIG. 7A, in constructing the allograft 8, cancellous wafer 28 is first placed on the cortical pin 40 by inserting an end 40a through the canal 28a, and sliding the cancellous wafer 28 onto the middle diameter 40b. Next, cancellous wafer 22 is placed on the cortical pin 40 by inserting an end 40a through the canal 22a, and sliding the cancellous wafer 22 onto the middle diameter 40b and adjacent cancellous wafer 28 such that groove 56 receives tab 46. Next, cortical member 24 slides onto the end portion 40a of the cortical pin 40 such that shoulder 40c rests on tab 42 once tab 42 is inserted into groove 44. Finally, cortical member 26 slides onto the opposite end portion 40a of the cortical pin 40 such that shoulder 40d rests within tab 58 of cancellous wafer 28, which is in turn surrounded by cortical wafer 26 due to the groove 48 receiving tab 58 of cancellous wafer 28.

As the allograft 8 is inserted into the surgically altered site of the patient in the plane of insertion I, the surgical mallet or other appropriate medical/surgical device is used to strike the allograft 8 in the direction of the plane of insertion I. Cortical wafer 26 and the end portion 40a of cortical pin 40 disposed within cortical wafer 26 absorb the initial energy imparted on the allograft 8. The energy imparted on the end portion 40a of the cortical pin 40 is transferred through shoulder 40d, which is surrounded by the stronger cortical bone tissue of cortical wafer 26, and into middle diameter 40b. From middle diameter 40b, the energy is transferred through the shoulder 40c of cortical pin 40, which is abutted against the hard cortical tab 42 of cortical member 24, thereby transferring the energy from cortical pin 40 to cortical wafer 24 and the end portion 40a of cortical pin 40 disposed within cortical wafer 24.

Referring to FIGS. 8 and 8A, an embodiment of the allograft 8 of the present invention comprising five wafers is disclosed. The allograft 8 has end cortical wafers 24 and 26 as described with respect to FIGS. 6, 6A, 7 and 7A hereinabove. The allograft 8 also has cancellous wafers 22 and 28 as described with respect to FIGS. 7 and 7A hereinabove. However, as shown in FIG. 8, an internal cortical wafer 80 is disposed adjacently between cancellous wafers 22 and 28. Cortical wafer 80 has a groove 68 which extends the length of cortical wafer 80 and snugly receives tab 46 of cancellous wafer 22. On the side of cortical wafer 80 opposite groove 68 is a tab 70 extending the length of cortical wafer 80, which is snugly received by groove 56 of cancellous wafer 28. Cortical wafer 80 has a canal 80a which is sufficiently sized to snugly receive middle diameter 30b of cortical pin 30. Cortical pin 30 is substantially the same as cortical pins 20 and 40 except that its middle diameter 30b is elongated sufficiently to snugly reside within cancellous wafers 22 and 28 and cortical wafer 80.

Referring to FIG. 8A, in constructing the allograft 8, cancellous wafer 28 is first placed on cortical pin 30 by inserting an end 30a through the canal 28a, and sliding the cancellous wafer 28 onto middle diameter 30b. Next, cortical wafer 80 slides onto the cortical pin 30 by inserting the same end 30a through the canal 80a, and sliding the cortical wafer 80 onto the middle diameter 30b and adjacent cancellous wafer 28 such that groove 56 receives tab 70. Next, cancellous wafer 22 is placed on the cortical pin 30 by inserting the same end 30a through the canal 22a, and sliding the cancellous wafer 22 onto the middle diameter 30b and adjacent cortical wafer 80 such that groove 68 receives tab 46. Next, cortical member 24 slides onto the end portion 30a of the cortical pin 30 such that shoulder 30c rests on tab 42 once tab 42 is inserted into groove 44. Finally, cortical member 26 slides onto the opposite end portion 30a of the cortical pin 30 such that shoulder 30d rests within tab 58 of cancellous wafer 28, which is in turn surrounded by cortical wafer 26 due to the groove 48 receiving tab 58 of cancellous wafer 28.

As the allograft 8 is inserted into the surgically altered site of the patient in the plane of insertion I, the surgical mallet or other appropriate medical/surgical device is used to strike the allograft 8 in the direction of the plane of insertion I. Cortical wafer 26 and the end portion 30a of cortical pin 30 disposed within cortical wafer 26 absorb the initial energy imparted on the allograft 8. The energy imparted on the end portion 30a of the cortical pin 30 is transferred through shoulder 30d, which is surrounded by the stronger cortical bone tissue of cortical wafer 26, and into middle diameter 30b. From middle diameter 40b, the energy is transferred through the shoulder 30c of cortical pin 30, which is abutted against the hard cortical tab 42 of cortical member 24, thereby transferring the energy from cortical pin 30 to cortical wafer 24 and the end portion 30a of cortical pin 30 disposed within cortical wafer 24.

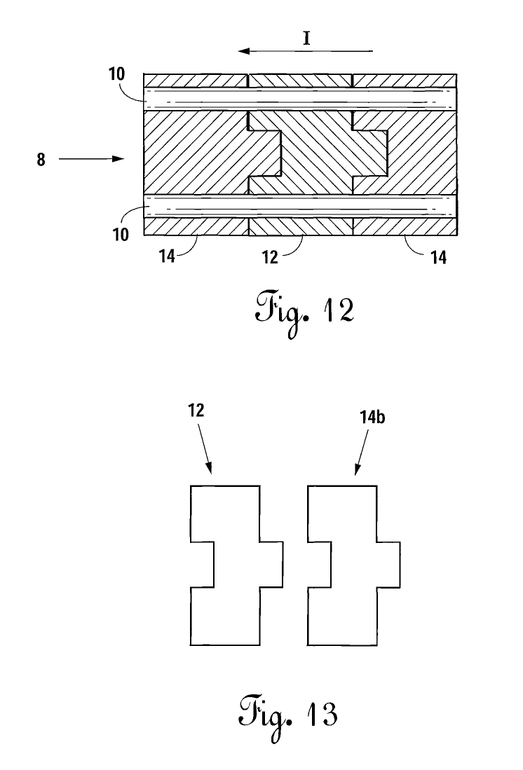

Referring to FIG. 11, another embodiment of the allograft 82 of the present invention is disclosed. In this embodiment, an anterior lumbar inner body fusion allograft 82 is disclosed. The allograft comprises a femoral ring 36 which is cut approximately in half. Inserted between the halves 36a and 36b of the femoral ring 36 is a cancellous ball 32. The cancellous ball is substantially spherically shaped. Extending from the cancellous ball 32 are cancellous spacers 34. The cancellous spacers 34 are pieces of cancellous bone which abut adjacently between the halves 36a and 36b of the femoral ring 34. The cancellous ball 32 and cancellous spacers 34 may be constructed of three separate pieces of cancellous bone wherein the spacers 34 are attached to the ball 32, or a single piece of cancellous bone.

Cortical pins 38 attach the two halves 36a and 36b to the cancellous spacers 34. The cortical pins 38 are shaped substantially similar to the rolling pin configuration of the cortical pins 20, 30 and 40 discussed hereinabove. Upon insertion of the allograft 82, the ends 38a of the cortical pins 38 receive the energy transferred from the strike of the surgical mallet on half 36b of femoral ring 36. The energy is transferred through shoulder 38d into middle diameter 38b, and onto shoulder 38c. Shoulder 38c abuts against half 36a of femoral ring 36. Therefore, the energy is transferred from shoulder 38c onto half 36a. The advantage of the cancellous spacers 34 and ball 32 is that it allows the allograft 82 made of the femoral ring 36 to be expanded such that larger allografts 82 can be assembled than is otherwise anatomically possible simply from retrieving a femoral ring 36 from a donor.

The allografts 8 of the present invention provide advantages not previously available in the art. Although the allografts 8 of the present invention have been described with reference to specific embodiments, this description is not meant to be construed in a limited sense. Various modifications of the disclosed embodiments, as well as alternative embodiments of the invention will become apparent to persons skilled in the art upon the reference to the description of the invention. For instance, it should be understood in the art that more than one cortical pin 20, 30, 40 could be inserted into the allografts 8 shown in FIGS. 1-8A, respectively to further secure the allograft 8 and transfer the energy during insertion.

Moreover, although described as three, four or five-wafer allografts 8, it should readily be understood that the allografts 8 of the present invention could have any number, composition and arrangement of cortical and cancellous wafers. In fact, it will be readily understood that the allografts 8 of the present invention can easily be sized by adding or removing internal cortical and/or cancellous wafers, or by increasing or decreasing the width of the wafers used in the allograft 8. It is furthermore desirable to insert an internal cortical wafer, such as that shown as cortical wafer 80 in FIGS. 8 and 8A where the width of any one or multiple cancellous wafers is approximately eight millimeters or more in order to add stability and load-bearing support to the allograft 8.

Furthermore, although described as being able to be inserted into the spinal column, or intervertebrally into a patient, the allografts 8 of the present invention can be inserted on or between any bone or bone segments where stabilization is required or desired. In light of the detailed description above, it is contemplated that the appended claims will cover such modifications that fall within the scope of the invention.

I claim:

1. A bone allograft for implanting into a surgically altered area of a human, said bone allograft comprising:
a first cortical bone member of a predefined size having a width, a length, and at least one canal extending though said width;
a first cancellous bone member of a predefined size having a width, a length, and at least one canal extending through said width;
a second cortical bone member of a predefined size having a width, a length, and at least one canal extending through said width;
at least one cortical pin extending through said canals of said (a) first cortical bone member, (b) first cancellous bone member, and (c) second cortical bone member;
wherein said cortical pin connects said first cortical bone member, said first cancellous bone member and said second cortical bone member to form said bone allograft such that said first cancellous bone member is disposed adjacently between said first cortical bone member and said second cortical bone member.

2. The bone allograft as recited in claim 1 wherein said at least one cortical pin further comprises:
a first end portion having a first diameter;
a middle portion adjacent said first end portion having a second diameter;
a second end portion adjacent said middle portion having a third diameter substantially the same as said first diameter;
wherein said first end portion is disposed within said canal in said first cortical bone member, said middle portion is disposed within said canal in said first cancellous bone member and said second end portion is disposed within said second cortical bone member; and
wherein said second diameter is greater than said first diameter and said third diameter.

3. The bone allograft as recited in claim 2 wherein:
said first cortical bone member further comprises a predefined notch along said length adjacent said first cancellous bone member;
said first cancellous bone member further comprises a predefined tab along said length adjacent said first cortical bone member and a predefined notch along said length adjacent said second cortical bone member;
said second cortical bone member further comprises a predefined tab along said length adjacent said first cancellous bone member; and
wherein said tab of said first cancellous bone member is disposed within said notch of said first cortical bone member and said tab of said second cortical bone member is disposed within said notch of said first cancellous bone member to form said bone allograft.

4. The bone allograft as recited in claim 3 wherein:
said canal of said first cortical bone member extends within said notch of said first cortical bone member;
said canal of said first cancellous bone member extends within said tab and said notch of said first cancellous bone member; and
said canal of said second cortical bone member extends within said tab of said second cortical bone member.

5. The bone allograft as recited in claim 2 further comprising:
a second cancellous bone member of a predefined size having a width, a length, and at least one canal extending through said width;
wherein part of said middle portion of said cortical pin is disposed within said canal of said second cancellous bone member; and
wherein said second cancellous bone member is disposed adjacently between said first cancellous bone member and said second cortical bone member.

6. The bone allograft as recited in claim 4 further comprising:
a second cancellous bone member of a predefined size having a width, a length, and at least one canal extending through said width, and further comprising a predefined tab along said length adjacent said first cancellous bone member and a predefined notch along said length adjacent said second cortical bone member;
wherein said second cancellous bone member is disposed adjacently between said first cancellous bone member and said second cortical bone member such that said tab of said second cortical bone member is disposed within said notch of said second cancellous bone member, and said tab of said second cancellous bone member is disposed within said notch of said first cancellous bone member; and
wherein part of said middle portion of said cortical pin is disposed within said canal of said second cancellous bone member.

7. The bone allograft as recited in claim 6 wherein said canal extends within said notch and said tab of said second cancellous bone member.

8. The bone allograft as recited in claim 5 further comprising:
a third cortical bone member of a predefined size having a width, a length, and at least one canal extending through said width;
wherein a part of said middle portion of said cortical pin is disposed within said canal; and
wherein said third cortical bone member is disposed adjacently between said first cancellous bone member and said second cancellous bone member.

9. The bone allograft as recited in claim 7 further comprising:
a third cortical bone member of a predefined size having a width, a length, and at least one canal extending through said width, and further comprising a predefined tab along said length adjacent said first cancellous bone member and a predefined notch along said length adjacent said second cancellous bone member;
wherein said third cortical bone member is disposed adjacently between said first cancellous bone member and said second cancellous bone member such that said tab of said third cortical bone member is disposed within said notch of said first cancellous bone member and said tab of said second cancellous bone member is disposed within said notch of said third cortical bone member to form said bone allograft; and
wherein a part of said middle portion of said cortical pin is disposed within said canal, said third cortical bone member being disposed adjacent said first cancellous bone member and said second cancellous bone member.

10. The bone allograft as recited in claim 9 wherein said canal extends within said notch and said tab of said third cortical bone member.

11. A bone allograft for implanting into a surgically altered area of a human, said bone allograft comprising:
a first cortical bone member of a predefined size having a width, a length, at least one canal extending through said width, and a predefined notch disposed along one side of said length;
at least one cancellous bone member of a predefined size having a width, a length, at least one canal extending through said width, a predefined tab along one side of said length adjacent said first cortical bone member, said tab being disposed within said notch of said first cortical bone member, and a notch disposed along the opposite side of said length;
a second cortical bone member of a predefined size having a width, a length, at least one canal extending through said width, and a predefined tab disposed along one side of said length adjacent said at least one cancellous bone member, said tab being disposed within said notch of said at least one cancellous bone member;
at least one cortical pin having a first end portion with a first diameter, a middle portion adjacent said first end portion with a second diameter, and a second end portion adjacent said middle portion with a third diameter substantially the same as said first diameter;
wherein said first end portion is substantially disposed within said canal of said first cortical bone member, said middle portion is substantially disposed within said canal of said at least one cancelleous bone member and said second end portion is substantially disposed within said canal of said second cortical bone member; and
wherein said second diameter is greater than said first diameter and said third diameter.

12. The bone allograft as recited in claim 11 further comprising:
a first cancellous bone member adjacent said first cortical bone member; and
a second cancellous bone member adjacently between said first cancellous bone member and said second cortical bone member such that said tab of said second cancellous bone member is disposed within said notch of said first cancellous bone member and said tab of said second cortical bone member is disposed within said notch of said second cancellous bone member.

13. The bone allograft as recited in claim 12 further comprising:
a third cortical bone member having a width, a length, at least one canal extending through said width, a predefined tab along said length adjacent said first cancellous bone member, and a predefined notch along said length adjacent said second cancellous bone member;
wherein said tab of said third cortical bone member is disposed within said notch of said first cancellous bone member and said tab of said second cancellous bone member is disposed within said notch of said third cortical bone member.

14. The bone allograft as recited in claim 13 wherein a part of said middle portion of said cortical pin is disposed within said canal of said third cortical bone member.

15. The bone allograft as recited in claim 11 comprising:
a plurality of cancellous bone members disposed between said first cortical bone member and said second cortical bone member;
a plurality of internal cortical bone members disposed adjacently between said plurality of cancellous bone members such that no more that two of said plurality of cancellous bone members are adjacent one another; and
wherein each of said plurality of cortical bone members comprises a width, and length, at least one canal extending through said width, a predefined tab along said length, said tab being disposed in said notch of an adjacent one of said plurality of cancellous bone members, and a notch along said length opposite said tab, said notch receiving a tab of another adjacent one of said plurality of cancellous bone members.

16. The bone allograft as recited in claim 15 wherein:
said canal of said first cortical bone member extends within said notch therein;
each canal of said plurality of cancellous bone members extends within each said notch and each said tab of said plurality of cancellous bone members;
each canal of said plurality of internal cortical bone members extends within each said notch and each said tab of said plurality of internal cortical bone members; and
said canal of said second cortical bone member extends within said tab therein.

17. The bone allograft as recited in claim 11 wherein:
said canal of said first cortical bone member extends within said notch therein;
said canal of said at least one cancellous bone members extends within each said notch and each said tab; and
said canal of said second cortical bone member extends within said tab therein.

18. A cortical bone pin for inserting into a plurality of bone wafers to form a bone allograft comprising:
a first cortical end portion having a first diameter;
a middle cortical portion adjacent said first end portion, said middle portion having a second diameter;
a second cortical end portion adjacent said middle portion and opposite said first end portion, said second end portion having a third diameter substantially the same as said first diameter of said first end portion;
a first shoulder at a junction of said first cortical end portion and said middle cortical portion;
a second shoulder at a junction of said middle cortical portion and said second cortical end portion; and
wherein said second diameter is greater than said first diameter and said third diameter.

19. The cortical bone pin as recited in claim 18 wherein said first cortical end portion, said middle cortical portion and said second cortical end portion are substantially cylindrically shaped.

20. The cortical bone pin as recited in claim 19 wherein said cortical bone pin is comprised of one piece of cortical bone.

21. The cortical bone pin as recited in claim 19 wherein said cortical bone pin is comprised of more than one piece of cortical bone.
